(19) United States
(12) Patent Application Publication
(10) Pub. No.: US 2023/0241020 A1
(43) Pub. Date: Aug. 3, 2023

(54) Scalp Numbing System

(71) Applicant: Cara Bonadies, Clifton, NJ (US)

(72) Inventor: Cara Bonadies, Clifton, NJ (US)

(21) Appl. No.: 18/161,402

(22) Filed: Jan. 30, 2023

Related U.S. Application Data
(60) Provisional application No. 63/304,731, filed on Jan. 31, 2022.

(51) Int. Cl. A61K 31/245; A61K 31/167

(52) U.S. Cl. CPC A61K 31/245; A61K 31/167; A61K 9/0014

(57) ABSTRACT

The present invention relates to a scalp numbing system comprised of at least one container that stores and/or can be used to apply the compound to a scalp before hair braiding/hair styling. In differing embodiments, the compound may be in a plurality of forms such as, but not limited to, a liquid, a spray, a foam, a gel, etc.

### CROSS-REFERENCE TO RELATED APPLICATION

The present application claims priority to, and the benefit of, U.S. Provisional Application No. 63/304,731, which was filed on Jan. 31, 2022, and is incorporated herein by reference in its entirety.

### FIELD OF THE INVENTION

The present invention relates generally to the field of hair products. More specifically, the present invention relates to a scalp numbing system comprised of at least one container that stores and/or can be used to apply the compound to a scalp before hair braiding/hair styling. In differing embodiments, the compound may be in a plurality of forms such as, but not limited to, a liquid, a spray, a foam, a gel, etc. Accordingly, the present disclosure makes specific reference thereto. Nonetheless, it is to be appreciated that aspects of the present invention are also equally applicable to other like applications, devices, and methods of manufacture.

### BACKGROUND

Hair braiding and hair styling can be a painful experience for many individuals. As a result, some individuals may refuse to sit in a styling chair and risk being in pain for the entire experience just to have their hair styled. In addition, the scalp can be incredibly tender after the styling process, and some individuals may suffer from serious headaches.

Therefore, there exists a long-felt need in the art for a product for hair braiding/hair styling. More specifically, there exists a long-felt need in the art for a scalp numbing system that can be used to numb the scalp of an individual whose hair is being braided or styled. In addition, there exists a long-felt need in the art for a scalp numbing system that can be applied to a scalp in a plurality of forms.

The subject matter disclosed and claimed herein, in one embodiment thereof, comprises a scalp numbing system. The system is comprised of at least one container that stores and/or can be used to apply the compound to a scalp before hair braiding/hair styling. In differing embodiments, the compound may be in a plurality of forms such as, but not limited to, a liquid, a spray, a foam, a gel, etc. In each embodiment, the container may be specifically designed to apply the specific form of the compound to a scalp.

In this manner, the scalp numbing system of the present invention accomplishes all of the foregoing objectives and provides a product for hair braiding/hair styling that can be used to numb the scalp of an individual whose hair is being braided or styled. Further, the compound of the system can be applied to a scalp in a plurality of forms. Various embodiments of the container can also allow the compound, in its various forms, to be applied to a scalp in a plurality of ways.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a scalp numbing system. The system is comprised of at least one container that stores and/or can be used to apply the compound to a scalp. In one embodiment, the container is intended to dispense the compound in a liquid form, wherein the container may be comprised of at least one lid with at least one spout and at least one removable cap. In this manner, the compound can be poured from the spout onto a scalp of a user before hair styling/braiding. Then, the compound can be washed from the scalp after hair styling/braiding has finished.

In another embodiment, the container is intended to dispense the compound in aerosol form. In this embodiment, the container is comprised of a body comprised of at least one lid and at least one nozzle having at least one trigger that can be depressed to draw the compound from the container and out the nozzle via at least one internal tubing. In this manner, the compound can be sprayed from the nozzle onto a scalp of a user before hair styling/braiding. Then, the compound can be washed from the scalp after hair styling/braiding has finished.

In a further embodiment, the container is intended to dispense the compound in a gel form. The container is comprised of a body comprised of at least one cap. The body is preferably deformable such that the compound can be squeezed out of the body to apply the compound to a scalp. The system may also be comprised of at least one applicator that aids a user in applying the gel compound to the scalp, wherein the applicator may be, but is not limited to, any style of brush known in the art. In this manner, the compound can be squeezed from the body onto a scalp of a user and can be further brushed onto the scalp via the applicator before hair styling/braiding. Then, the compound can be washed from the scalp after hair styling/braiding has finished.

In an additional embodiment, the container is intended to dispense the compound in a foam form. The container is comprised of a body comprised of at least one lid with at least one nozzle, wherein the lid can be depressed to draw the compound from the container and out the nozzle via at least one internal tubing. In this manner, the compound can be sprayed from the nozzle onto a scalp of a user before hair styling/braiding. Then, the compound can be washed from the scalp after hair styling/braiding has finished.

Accordingly, the scalp numbing system of the present invention is particularly advantageous as it provides a product for hair braiding/hair styling that can be used to numb the scalp of an individual whose hair is being braided or styled. Further, the compound of the system can be applied to a scalp in a plurality of forms. In addition, various embodiments of the container can also allow the compound, in its various forms, to be applied to a scalp in a plurality of ways. In this manner, the scalp numbing system overcomes the limitations of existing methods of braiding and styling hair known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a user using a scalp numbing system of the present invention in accordance with the disclosed architecture;

FIG. 2 illustrates a graphical view of a compound of one potential embodiment of a scalp numbing system of the present invention in accordance with the disclosed architecture;

FIG. 3 illustrates a front view of multiple potential embodiments of a container of one potential embodiment of a scalp numbing system of the present invention in accordance with the disclosed architecture;

FIG. 4 illustrates a flowchart of a first method of using one potential embodiment of a scalp numbing system of the present invention in accordance with the disclosed architecture;

FIG. 5 illustrates a flowchart of a second method of using one potential embodiment of a scalp numbing system of the present invention in accordance with the disclosed architecture;

FIG. 6 illustrates a flowchart of a third method of using one potential embodiment of a scalp numbing system of the present invention in accordance with the disclosed architecture; and

FIG. 7 illustrates a flowchart of a fourth method of using one potential embodiment of a scalp numbing system of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a product for hair braiding/hair styling. More specifically, there exists a long-felt need in the art for a scalp numbing system that can be used to numb the scalp of an individual whose hair is being braided or styled. In addition, there exists a long-felt need in the art for a scalp numbing system that can be applied to a scalp in a plurality of forms.

The present invention, in one exemplary embodiment, is comprised of a scalp numbing system comprised of at least one container that stores and can be used to apply the compound to a scalp before hair braiding/hair styling. In differing embodiments, the compound may be in a plurality of forms such as, but not limited to, a liquid, a spray, a foam, a gel, etc. In each embodiment, the container may be specifically designed to apply the specific form of the compound to a scalp.

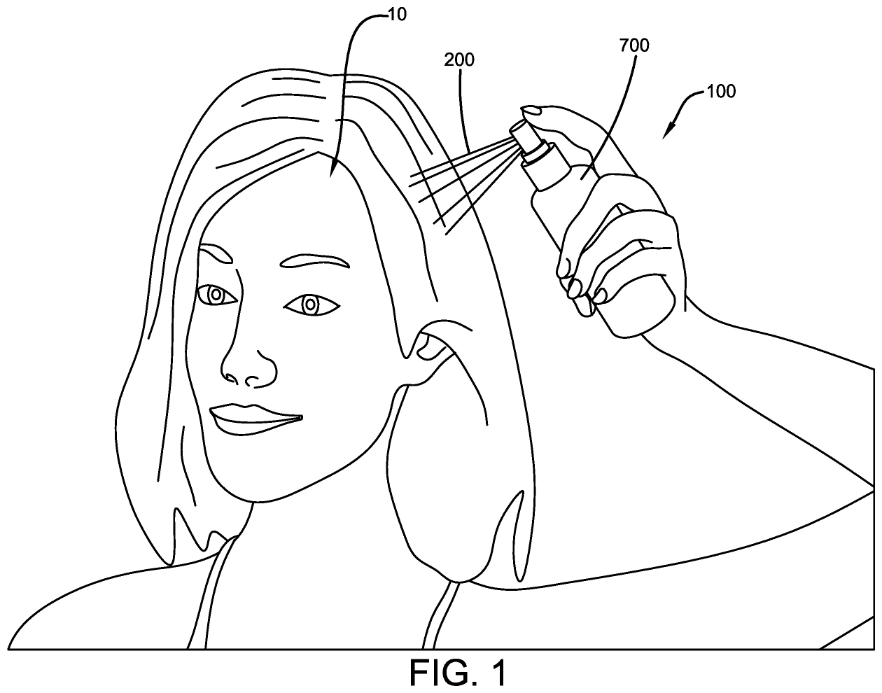

Referring initially to the drawings, FIG. 1 illustrates a perspective view of one potential embodiment of a user using the scalp numbing system 100 of the present invention in accordance with the disclosed architecture. The system 100 is comprised of a compound 200 comprised of at least one ingredient 210 and at least one method of use 300. The compound 200 is designed to numb the scalp of the user during the hair styling process. In differing embodiments, the compound 200 may be in a plurality of forms such as, but not limited to, a liquid, a foam, a gel, etc.

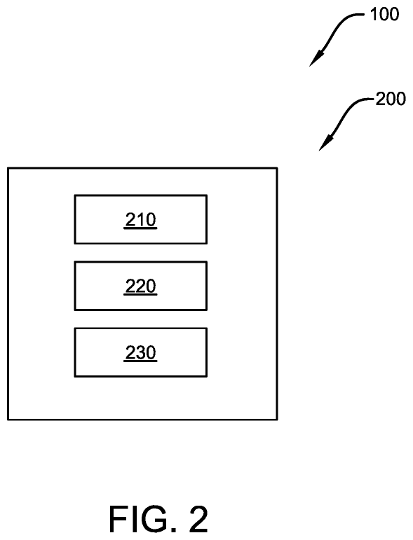

As seen in FIG. 2, the compound 200 may be made from a plurality of ingredients in differing embodiments with respect to whether the compound 200 is a liquid, aerosol, gel, foam, pomade, or another form. However, all embodiments of the compound 200 are preferably comprised of a first ingredient 210 of 20% benzocaine, a second ingredient 220 of 4% lidocaine for numbing, or a similar amount of pramoxine, dibucaine, and/or tetracaine for numbing as a third ingredient 230.

In a liquid embodiment, the compound 200 is comprised of 20% benzocaine and 4% lidocaine for numbing. The remaining 76% may be comprised of any combination of pramoxine, dibucaine, tetracaine, benzyl alcohol, carbopol, lecithin, propylene glycol, vitamin E acetate, and a purified water base, etc., or other similar compounds of the like commonly found in numbing creams.

In a liquid and aerosol embodiment, the compound 200 is comprised of 20% benzocaine and 4% lidocaine for numbing. The remaining 76% may be comprised of any combination of pramoxine, dibucaine, tetracaine, benzyl alcohol, carbopol, lecithin, propylene glycol, vitamin E acetate, and a purified water base.

In a gel embodiment, the compound 200 is comprised of 20% benzocaine and 4% lidocaine for numbing. The remaining 76% may be comprised of any combination of pramoxine, dibucaine, tetracaine, benzyl alcohol, carbopol, lecithin, propylene glycol, vitamin E acetate, and a gel base.

In a pomade embodiment, the compound 200 is comprised of 20% benzocaine and 4% lidocaine for numbing. The remaining 76% may be comprised of any combination of pramoxine, dibucaine, tetracaine, benzyl alcohol, carbopol, lecithin, propylene glycol, vitamin E acetate, and a pomade base.

In a foam embodiment, the compound 200 is comprised of 20% benzocaine and 4% lidocaine for numbing. The remaining 76% may be comprised of any combination of pramoxine, dibucaine, tetracaine, benzyl alcohol, carbopol, lecithin, propylene glycol, vitamin E acetate, and a foam base.

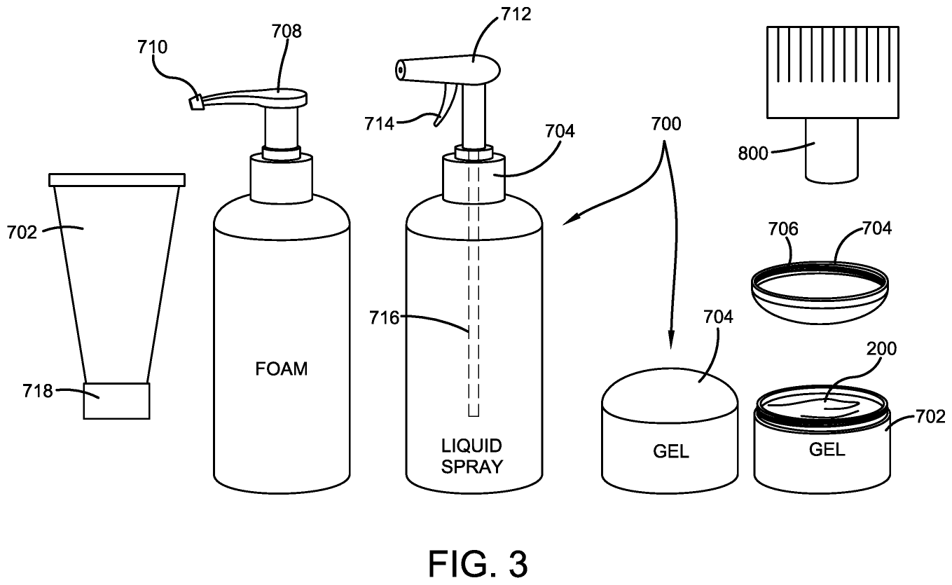

FIG. 3 illustrates a front view of multiple potential embodiments of a container 700 of one potential embodiment of a scalp numbing system 100 of the present invention in accordance with the disclosed architecture. The system 100 is comprised of at least one container 700 that stores and/or can be used to apply the compound 200 to a scalp 10. The container 700 and all components may be made from a durable metal material such as, but not limited to, stainless steel or aluminum. The container 700 may also be made from a plastic material such as, but not limited to, acrylic, polycarbonate, polyethylene, thermoplastic, acrylonitrile butadiene styrene, low-density polyethylene, medium-density polyethylene, high-density polyethylene, polyethylene terephthalate, polyvinyl chloride, polystyrene, polylactic acid, acetal, nylon, fiberglass, recycled plastic, biodegradable plastic, etc.

In one embodiment, the container 700 is intended to dispense the compound 200 in a liquid form. The container 700 may be comprised of at least one lid 704 that removably attaches to the body 702 via at least one fastener 706 such as, but not limited to, reciprocating threads, magnets, clips, etc. The lid 704 may further be comprised of at least one spout 708 with at least one removable cap 710. In this manner, the compound 200 can be poured from the spout 708 onto a scalp 10 of a user.

In another embodiment, the container 700 is intended to dispense the compound 200 in aerosol form. The container 700 is comprised of a body 702 comprised of at least one lid 704 with at least one nozzle 712 having at least one trigger 714 that can be depressed to draw the compound 200 from the container 700 and out the nozzle 712 via at least one internal tubing 716. The nozzle 712 may be turned to allow a user to adjust the size, intensity, and/or spray pattern of the compound 200 leaving the nozzle 712 in aerosol form and/or to prevent any compound 200 from leaving the container 700. In this manner, the compound 200 can be sprayed from the nozzle 712 onto a scalp 10 of a user.

In a further embodiment, the container 700 is intended to dispense the compound 200 in a gel form. The container 700 is comprised of a body 702 comprised of at least one cap 718. The cap 718 removably attaches to the body 702 via at least one fastener 706 such as, but not limited to, reciprocating threads, magnets, clips, etc. The cap 718 may also be a snap cap in one embodiment. The body 702 is preferably deformable such that the compound 200 can be squeezed out of the body 702 to apply the compound 200 to a scalp 10. In this embodiment, the system 100 may be comprised of at least one applicator 800 that aids a user in applying the gel compound 200 to the scalp 10, wherein the applicator 800 may be, but is not limited to, any style of brush known in the art. In this manner, the compound 200 can be squeezed from the body 702 onto a scalp 10 of a user and can be further brushed onto the scalp 10 via the applicator 800.

In an additional embodiment, the container 700 is intended to dispense the compound 200 in a foam form. The container 700 is comprised of a body 702 comprised of at least one lid 704 with at least one nozzle 712, wherein the lid 704 can be depressed to draw the compound 200 from the container 700 and out the nozzle 712 via at least one internal tubing 716. The nozzle 712 may be turned to allow a user to adjust the size, intensity, and/or spray pattern of the compound 200 leaving the nozzle 712 in foam form and/or prevent any compound 200 from leaving the container 700. In this manner, the compound 200 can be sprayed from the nozzle 712 onto a scalp 10 of a user.

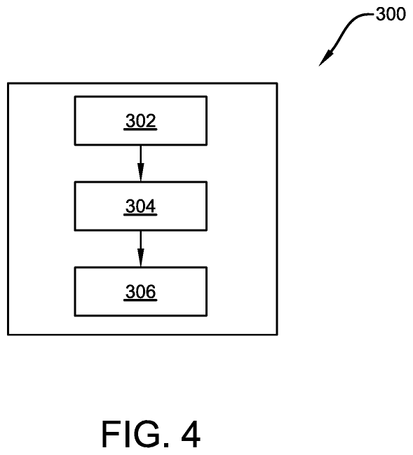

As seen in FIG. 4, in the first method of use 300, the compound 200 is in the form of a liquid. First, the compound 200 can be poured from a container 700 onto a scalp 10 of a user at a period before hair braiding/hair styling is to begin [Step 302]. The period is preferably (but not limited to) at least five minutes before hair braiding/hair styling is to begin. Then, the compound 200 numbs the scalp 10 [Step 304]. In this manner, a user does not experience pain during the braiding/styling process. After braiding/styling, the compound 200 can be washed from the scalp 10 [Step 306].

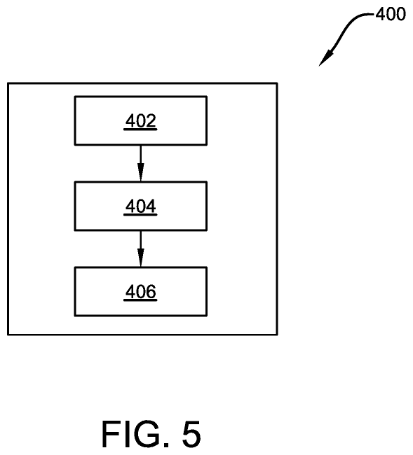

As seen in FIG. 5, in the second method of use 400, the compound 200 is in the form of a foam. First, the compound 200 can be sprayed from a container 700 onto a scalp 10 of a user at a period before hair braiding/hair styling is to begin [Step 402]. The period is preferably (but not limited to) at least five minutes before hair braiding/hair styling is to begin. Then, the compound 200 numbs the scalp 10 [Step 404]. In this manner, a user does not experience pain during the braiding/styling process. After braiding/styling, the compound 200 can be washed from the scalp 10 [Step 406].

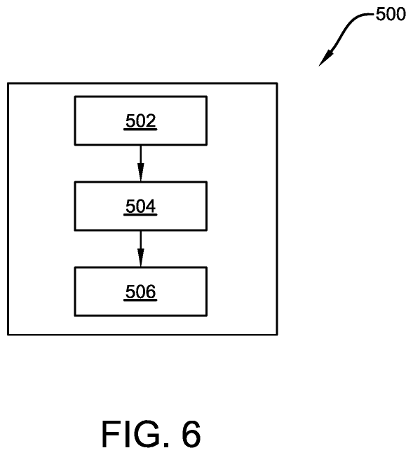

As seen in FIG. 6, in the third method of use 500, the compound 200 is in the form of an aerosol spray. First, the compound 200 can be sprayed from a container 700 onto a scalp 10 of a user at a period before hair braiding/hair styling is to begin [Step 502]. The period is preferably (but not limited to) at least five minutes before hair braiding/hair styling is to begin. Then, the compound 200 numbs the scalp 10 [Step 504]. In this manner, a user does not experience pain during the braiding/styling process. After braiding/styling, the compound 200 can be washed from the scalp 10 [Step 506].

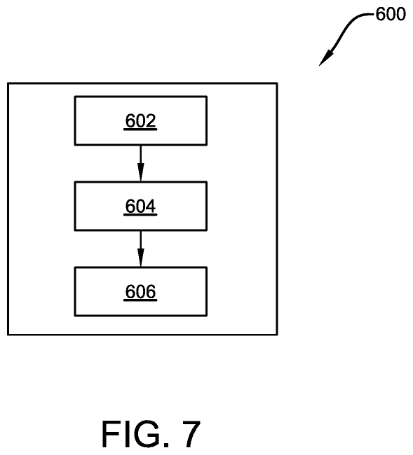

As seen in FIG. 7, in the fourth method of use 600, the compound 200 is in the form of a gel that is preferably clear. First, the compound 200 can be applied onto a scalp 10 of a user at a period before hair braiding/hair styling is to begin using at least one applicator 800 [Step 602]. The period is preferably (but not limited to) at least five minutes before hair braiding/hair styling is to begin. Then, the compound 200 numbs the scalp 10 [Step 604]. In this manner, a user does not experience pain during the braiding/styling process. After braiding/styling, the compound 200 can be washed from the scalp 10 [Step 606].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “scalp numbing system” and “system” are interchangeable and refer to the scalp numbing system 100 of the present invention.

Notwithstanding the foregoing, the scalp numbing system 100 of the present invention and its various components can be of any suitable size and configuration as is known in the art without affecting the overall concept of the invention, provided that they accomplish the above-stated objectives. One of ordinary skill in the art will appreciate that the size, configuration, and material of the scalp numbing system 100 as shown in the FIGS. are for illustrative purposes only, and that many other sizes and shapes of the scalp numbing system 100 are well within the scope of the present disclosure. Although the dimensions of the scalp numbing system 100 are important design parameters for user convenience, the scalp numbing system 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A scalp numbing system comprising:
a compound comprised of a first ingredient, a second ingredient and a third ingredient.

2. The scalp numbing system of claim 1, wherein the first ingredient is comprised of a benzocaine.

3. The scalp numbing system of claim 2, wherein the compound is comprised of 20% benzocaine.

4. The scalp numbing system of claim 1, wherein the second ingredient is comprised of a lidocaine.

5. The scalp numbing system of claim 4, wherein the compound is comprised of 4% lidocaine.

6. The scalp numbing system of claim 1, wherein the third ingredient is comprised of a pramoxine, a dibucaine, a tetracaine, a benzyl alcohol, a larbopol, a lecithin, a propylene glycol, a vitamin E acetate, a gel base, a pomade base, a foam base, or a purified water base.

7. A scalp numbing system comprising:
a compound comprised of a first ingredient, a second ingredient and a third ingredient; and
a container.

8. The scalp numbing system of claim 7, wherein the first ingredient is comprised of a benzocaine.

9. The scalp numbing system of claim 8, wherein the compound is comprised of 20% benzocaine.

10. The scalp numbing system of claim 7, wherein the second ingredient is comprised of a lidocaine.

11. The scalp numbing system of claim 10, wherein the compound is comprised of 4% lidocaine.

12. The scalp numbing system of claim 7, wherein the third ingredient is comprised of a pramoxine, a dibucaine, a tetracaine, a benzyl alcohol, a carbopol, a lecithin, a propylene glycol, a vitamin E acetate, a gel base, a pomade base, a foam base, or a purified water base.

13. The scalp numbing system of claim 7, wherein the compound is comprised of a liquid form, a foam form, a gel form, a pomade form, or an aerosol form.

14. A method of using a scalp numbing system, the method comprising the steps of:
applying a compound of a scalp numbing system to a scalp of a user for a first period of time;
allowing the compound to remain on the scalp for a second period of time; and
washing the compound from the scalp after a third period of time.

15. The method of claim 14, wherein the compound is applied to the scalp before a hair of the user is braided.

16. The method of claim 14, wherein the compound is washed from the scalp after the hair of the user is braided.

17. The method of claim 14, wherein the compound is applied to the scalp from a container.

18. The method of claim 14, wherein the container is comprised of a liquid form, a foam form, a gel form, a pomade form, or an aerosol form.

19. The method of claim 18, wherein the container is comprised of a nozzle.

20. The method of claim 18, wherein the compound numbs the scalp of the user.
